(19) United States
(12) Patent Application Publication
(10) Pub. No.: US 2017/0007430 A1
(43) Pub. Date: Jan. 12, 2017

(54) METHOD FOR IMPROVING STENT RETENTION AND DEPLOYMENT CHARACTERISTICS

(71) Applicant: BOSTON SCIENTIFIC SCIMED, INC., MAPLE GROVE, MN (US)

(72) Inventors: PAUL RICHARD STRAUMANN, MAPLE GROVE, MN (US); AMOD SHRIDHAR MODAK, MAPLE GROVE, MN (US); RACHEL JAPUNTICH, ROSEVILLE, MN (US)

(73) Assignee: BOSTON SCIENTIFIC SCIMED, INC., MAPLE GROVE, MN (US)

(21) Appl. No.: 15/198,716

(22) Filed: Jun. 30, 2016

Related U.S. Application Data
(60) Provisional application No. 62/191,098, filed on Jul. 10, 2015.

(51) Int. Cl. A61F 2/90; B29C 65/44; B29C 65/02; A61F 2/844; A61F 2/958

(52) U.S. Cl. CPC A61F 2/90; A61F 2/844; A61F 2/958; B29C 65/02; B29C 65/44; A61F 2250/0067; A61F 2210/0004; A61F 2002/9522

(57) ABSTRACT

Medical devices and methods for making, preparing, and using medical devices are disclosed. An example method may include disposing an implantable medical device along an outer surface of a balloon. The implantable medical device may include a polymeric stent having a plurality of openings formed therein. The implantable medical device may be designed to shift between a compressed configuration having a compressed diameter and an expanded configuration having an expanded diameter. The method may also include compressing the implantable medical device to an intermediate diameter between the compressed diameter and the expanded diameter and applying inflation pressure to the balloon so that at least a portion of the balloon extends at least partially into at least one of the plurality of openings in the implantable medical device.

### CROSS-REFERENCE TO RELATED APPLICATIONS

This application claims priority under 35 U.S.C. §119 to U.S. Provisional Application Ser. No. 62/191,098, filed Jul. 10, 2015, the entirety of which is incorporated herein by reference.

### TECHNICAL FIELD

The present disclosure pertains to medical devices, and methods for manufacturing and/or preparing medical devices.

### BACKGROUND

A wide variety of intracorporeal medical devices have been developed for medical use, for example, intravascular use. Some of these devices include guidewires, catheters, stents, delivery systems, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices. An example embodiment method may include a method for preparing a medical device. The method comprises:

disposing an implantable medical device along an outer surface of a balloon;

wherein the implantable medical device includes a polymeric stent having a plurality of openings formed therein;

wherein the implantable medical device is designed to shift between a compressed configuration having a compressed diameter and an expanded configuration having an expanded diameter;

compressing the implantable medical device to an intermediate diameter between the compressed diameter and the expanded diameter; and

applying inflation pressure to the balloon so that at least a portion of the balloon extends at least partially into at least one of the plurality of openings in the implantable medical device.

Alternatively or additionally to the embodiment above, further comprising expanding the implantable medical device to a diameter larger than a manufactured diameter of the implantable medical device before compressing the implantable medical device to the intermediate diameter.

Alternatively or additionally to any of the embodiments above, further comprising applying heat to the implantable medical device.

Alternatively or additionally to any of the embodiments above, applying heat to the implantable medical device includes heating at a temperature of about 30° C. to about 80° C.

Alternatively or additionally to any of the embodiments above, applying heat to the implantable medical device includes heating at a temperature above the glass transition temperature (T_{g}) of the balloon.

Alternatively or additionally to any of the embodiments above, applying heat to the implantable medical device includes applying heat while compressing the implantable medical device.

Alternatively or additionally to any of the embodiments above, applying inflation pressure to the balloon occurs while compressing the implantable medical device.

Alternatively or additionally to any of the embodiments above, applying inflation pressure to the balloon occurs after compressing the implantable medical device.

Alternatively or additionally to any of the embodiments above, further comprising compressing the implantable medical device to a second intermediate diameter that is smaller than the intermediate diameter.

Alternatively or additionally to any of the embodiments above, the implantable medical device is a bioabsorbable polymeric stent, a drug coated polymeric stent, or a drug coated bioabsorbable polymeric stent.

Alternatively or additionally to any of the embodiments above, applying inflation pressure to the balloon includes applying about 10 psi to about 60 psi of inflation pressure.

A medical device system is disclosed. The system comprises:

an elongate shaft having a distal end region;

an expandable balloon coupled to the distal end region;

a stent secured to the balloon, wherein the stent has a plurality of openings formed therein;

wherein the stent is a polymeric stent, a bioabsorbable metal stent, or a drug coated metal stent; and

wherein at least a portion of the balloon extends at least partially into at least one of the plurality of openings in the stent.

Alternatively or additionally to any of the embodiments above, the stent is a bioabsorbable polymeric stent.

Alternatively or additionally to any of the embodiments above, the stent is a drug coated polymeric stent.

Alternatively or additionally to any of the embodiments above, the stent is a drug coated bioabsorbable polymeric stent.

Alternatively or additionally to any of the embodiments above, the balloon has a distal balloon cone and a proximal balloon cone, and wherein a ledge is formed in the balloon adjacent to the distal balloon cone, the proximal balloon cone, or both, wherein the ledge is configured to enhance securement of the stent to the balloon.

A method for preparing a medical device is disclosed. The method comprises:

disposing a stent along an outer surface of a balloon;

wherein the stent includes a plurality of openings;

wherein the stent is capable of shifting between a fully compressed configuration having a fully compressed diameter and an expanded configuration having an expanded diameter;

wherein the stent has a manufactured diameter that is between the fully compressed diameter and the expanded diameter;

expanding the stent from the manufactured diameter to a first intermediate diameter;

compressing the stent from the first intermediate diameter to a second intermediate diameter, the second intermediate diameter being smaller than the manufactured diameter and being between the fully compressed diameter and the expanded diameter;

applying inflation pressure to the balloon so that one or more sections of the balloon extend through at least some of the plurality of openings in the stent; and

applying heat to the stent while compressing the stent, while applying inflation pressure to the balloon, or while compressing the stent and applying inflation pressure to the balloon.

Alternatively or additionally to any of the embodiments above, applying heat to the stent includes applying heat while compressing the stent.

Alternatively or additionally to any of the embodiments above, further comprising compressing the stent to a third intermediate diameter that is smaller than the second intermediate diameter.

Alternatively or additionally to any of the embodiments above, the stent is a metal stent, a bioabsorbable metal stent, a drug coated metal stent, or a drug coated bioabsorbable metal stent.

Alternatively or additionally to any of the embodiments above, the stent is a polymeric stent, a bioabsorbable polymeric stent, a drug coated polymeric stent, or a drug coated bioabsorbable polymeric stent.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 schematically illustrates an example medical device system;

FIG. 2 is a cross-sectional view of a portion of an example medical device system;

FIG. 3 is a side view of an example stent;

FIG. 4 is a cross-sectional view of a portion of an example medical device system;

FIG. 4A is a graph illustrating an example crimping process;

FIG. 5 is a cross-sectional view of a portion of an example stent and a balloon; and

FIG. 6 is a side view of an example stent.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g., 1 inch to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

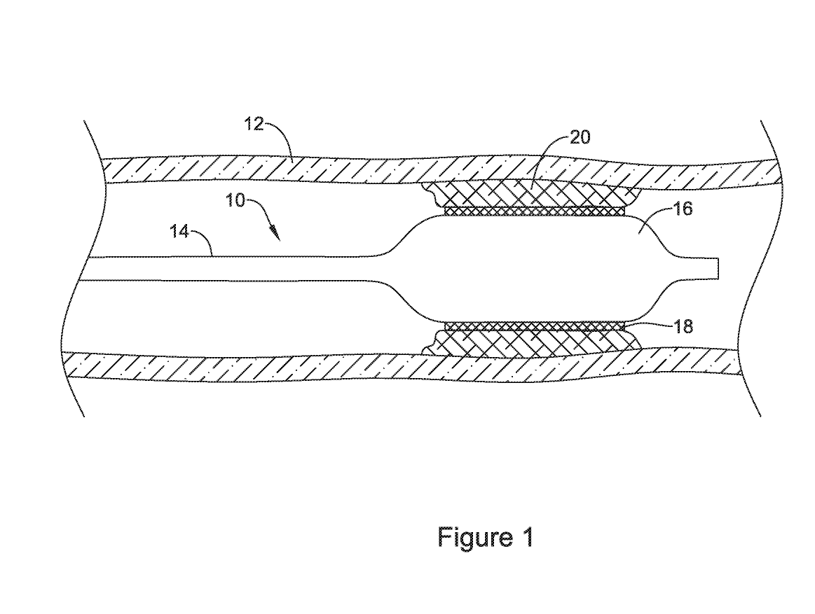

FIG. 1 schematically illustrates an example medical device system 10 (system) disposed within a blood vessel 12. System 10 may take the form of a stent delivery system including a catheter shaft 14, an expandable member or balloon 16 coupled to shaft 14, and an implantable medical device 18 coupled to balloon 16. In this example, implantable medical device 18 is a stent that may be used to treat a lesion 20. Other implantable medical devices are contemplated. In addition, other systems are contemplated that may be designed to be used in a variety of body lumens including, but not limited to, coronary blood vessels, peripheral blood vessels, along the pancreatic and/or biliary tract, along an airway, along the urinary tract, or the like.

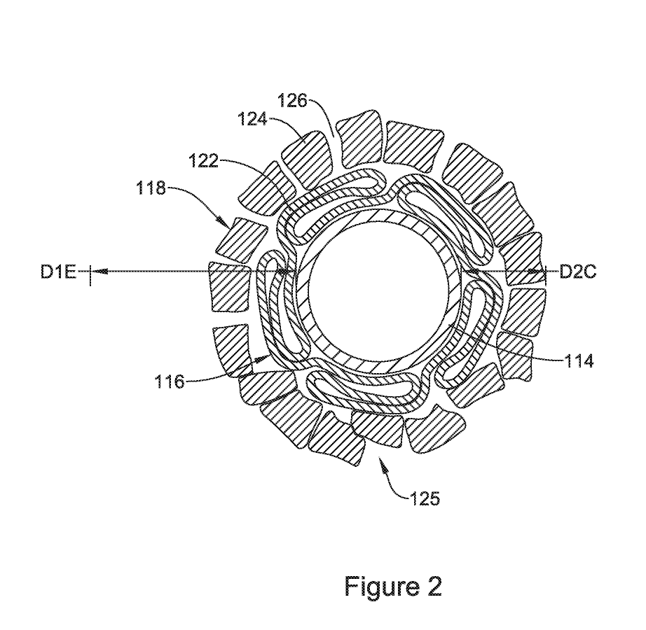

Stents, for example balloon expandable stents, are typically secured to the balloon of a balloon catheter. The processes for securing the stent to the balloon may involve a single stage diameter reduction or crimping step where the stent is compressed onto the balloon in one quick movement to a set force. FIG. 2 is a partial cross-sectional view of an example stent 118 crimped onto balloon 116 using a single step crimping process. The balloon 116 may be coupled to catheter shaft 114. Here it can be seen that stent 118 may include a plurality of struts 124. Openings 126 may be defined between struts 124. Stent 118 may be compressed from a first “expanded” diameter D1E to a second “compressed” diameter D2C. For the purposes of this disclosure, the first fully expanded diameter D1E may be understood as the diameter that stent 118 is designed to be fully expanded to. In use, stent 118 may be deployed at a diameter that is slightly less that the fully expanded diameter D1E due to, for example, the shape of the target anatomy, intravascular debris/lesions, etc. In at least some instances, the fully expanded diameter D1E corresponds to the diameter of stent 118 prior to being crimped. For the purposes of this disclosure, the fully compressed diameter D2C may be understood as the diameter that stent 118 is designed to be compressed to. In at least some instances, the compressed diameter D2C corresponds to the diameter of stent 118 when it is crimped onto balloon 116.

Balloon 116 may also be folded into a compressed configuration and may define a plurality of folds 122. In some instances, folds 122 may be described as wings. Folds 122 may include overlapping sections of balloon 116. This may include a single overlapping layer or multiple overlapping layers. It can be appreciated that, in at least some instances, the compressed diameter D2C corresponds to the diameter of stent 118 when it is crimped onto the folded balloon 116.

In some cases, it may be desired for a stent to be crimped less than all the way down on to the balloon. For example, some polymeric and/or bioabsorbable stents may utilize wider struts in order to achieve comparable radial strength to metal stents, because of, for example, material differences. Wider struts may increase the minimum crimp diameter that a stent can achieve before struts collide in an undesirable way. In an example, the minimum crimp diameter of a stent may be greater than the compressed diameter D2C. Some previous crimping processes (e.g., single step crimping processes) may take the stent below its minimum crimp diameter, which can cause stent damage or loss of critical mechanical properties, such as radial strength. In FIG. 2, which may be representative of a polymer stent that is crimped below its minimum crimping diameter, strut twisting and deformation can be seen (e.g., the bottom struts at reference number 125 in FIG. 2).

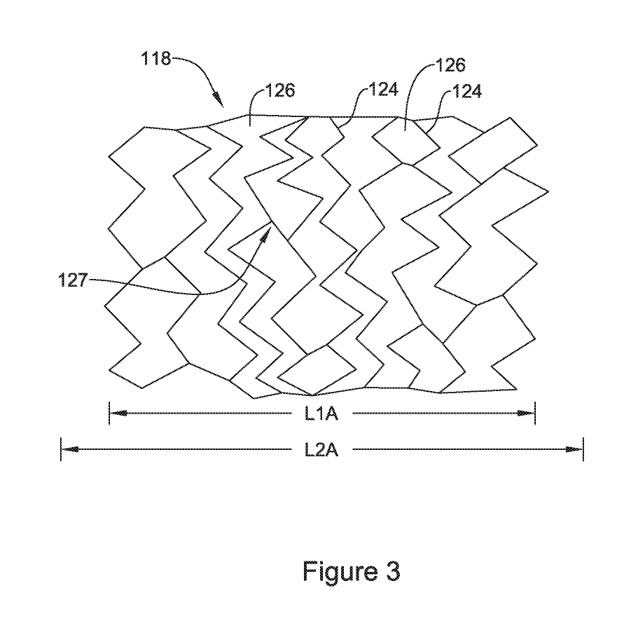

For larger diameter metal stents (e.g., about 4.50 mm to about 5.00 mm), higher foreshortening (e.g., stent length after deployment minus the stent length before deployment) can be seen during deployment due to the large difference in crimped diameter and deployed stent diameter. In at least some instances, the ends of the balloon may open before the middle of the stent, creating a large diameter gradient within the stent, which can cause the end strut rows to slide towards the middle of the stent as the balloon opens, to the point where several of the end strut rows may overlap each other (e.g., at reference number 127 in FIG. 2) and shorten the deployed length of the stent. For example, FIG. 3 schematically illustrates stent 118, including struts 124 and openings 126, in a deployed configuration. As shown in FIG. 3, stent 118 may be foreshortened such that the length L1A of stent 118 in an expanded configuration is shorter than the expanded length L2A (e.g., where the expanded length L2A is a length corresponding to the length that stent 118 is designed to elongate to when expanded), such as a result of end strut rows overlapping each other 127. In other words, the difference between length L2A and L1A may be relatively large.

It may be desirable to limit the amount of foreshortening of stent 118 (and/or other stents disclosed herein). A potential way to reduce stent deformation and/or reduce foreshortening may include crimping the stent to a larger diameter, such as an intermediate diameter between the fully expanded diameter D1E and the fully compressed diameter D2C. This may reduce stent deformation and preserve more of the stent's as-cut mechanical properties, in the case of polymeric stents. However, crimping to a larger diameter may impact stent securement. In other words, a portion of the stent may embolize or shift along the balloon during delivery and/or deployment. It may be desirable to reduce the amount of migration of the stent and/or improve the securement of the stent to the balloon in order to mitigate migration while also controlling for stent deformation and limiting foreshortening.

Reduced foreshortening, reduced stent migration, and/or improved securement of the stent may be achieved through crimping and/or stent securement processes, such as those processes disclosed herein. For example, as described herein, the stent may be retained on the balloon by the plastic deformation of the stent or by the stent becoming partially embedded into the balloon. Stent retention forces may result from the combination of obstructive forces due to balloon material ridges forming around elements of the stent, or residual normal force on the stent from the compressed balloon layers. Heated crimping elements, in one or more embodiment, may also be used to soften the balloon material to allow the stent to emboss or embed into the balloon more readily. In various embodiments, a balloon inflation step is used before and/or during the crimp cycle to form the balloon up against the stent and in-between stent struts for additional securement and stent to balloon engagement. Some additional details of at least some of the crimping processes contemplated herein are discussed below.

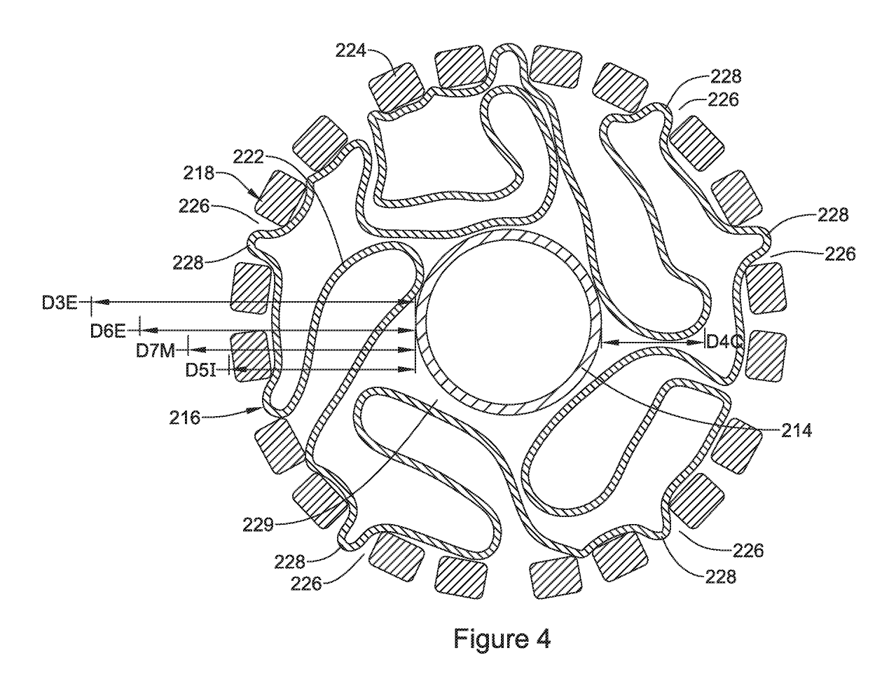

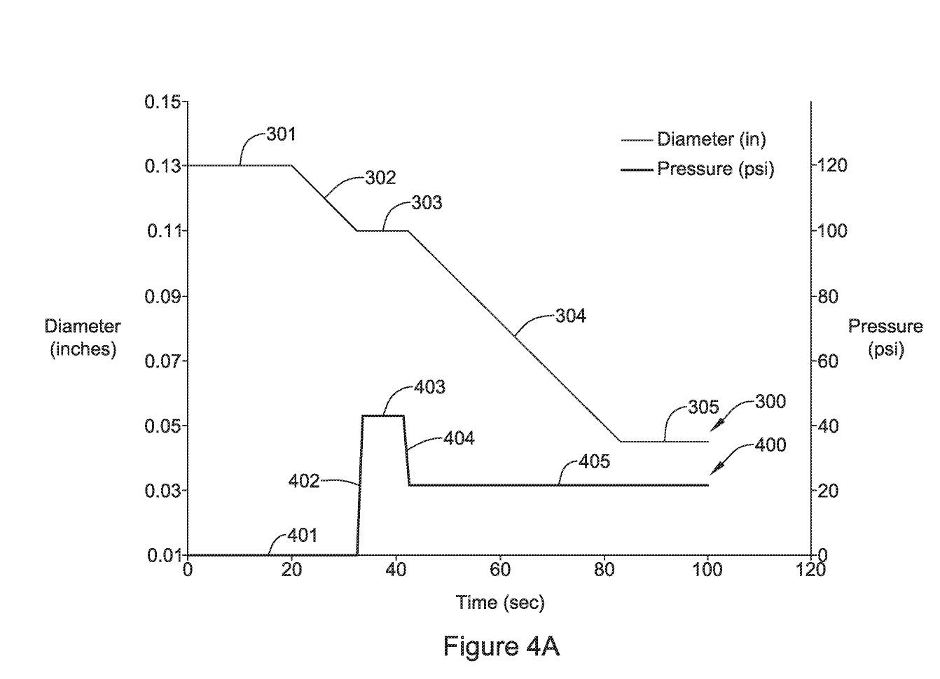

FIG. 4 is a partial cross-sectional view of an example stent 218 crimped onto balloon 216 using a multiple step crimping process that is designed to lessen stent deformation, control for higher foreshortening, or improve securement of stent 218 to balloon 216. Also shown in FIG. 4 is catheter shaft 214. Balloon 216 may be folded and define a plurality of folds 222. Stent 218 may include a plurality of struts 224. Openings 226 may be defined between struts 224. Stent 218 may be crimped onto balloon 216. Like stent 118 described in relation to FIG. 2, stent 218 may be capable of shifting between a first larger diameter D3E to a second smaller diameter D4C. In at least some instances, diameter D3E and diameter D4C may correspond to the largest and smallest diameters, respectively, that stent 218 may be expanded/compressed to without being damaged/deformed.

In some instances, stent 218 may be compressed to an intermediate diameter D5I. In other instances, stent 218 may be expanded to an expanded diameter D6E through balloon inflation before being compressed. More particularly, stent 218 may be manufactured or otherwise formed to have a “manufactured diameter” D7M and then may be expanded prior to the crimping process (e.g., to diameter D6E). Before, during, and/or after expansion or compression, inflation pressure may be applied to balloon 216. In an example, a portion 228 of balloon 216 may extend at least partially into or through an opening 226. In other words, balloon 216 may be formed up against and through at least one of the plurality of openings 226 between struts 224 to provide a stent to balloon interaction and stent retention at larger diameters. The processes disclosed herein where portions 228 of balloon 216 extend at least partially into or through at least one of the plurality of openings 226 may be termed “pillowing” due to, for example, stent 218 being held or “pillowed” by balloon 216.

In some instances, stent 218 may undergo an initial compression. In other instances, stent 218 may undergo an initial expansion through balloon inflation. Stent 218 may undergo one or more diameter reduction steps where stent 218 is compressed to an intermediate diameter (e.g., D5I) and inflation pressure is applied to balloon 216. For example, stent 218 may be a polymeric stent that has a manufactured diameter of about 0.05 inches to about 0.2 inches, or about 0.1 inches to about 0.15 inches, or about 0.13 inches and can be compressed to an intermediate diameter D5I of about 0.02 inches to about 0.15 inches, or about 0.04 inches to about 0.06 inches, or about 0.051 inches. In some instances, stent 218 may undergo multiple compressions. Inflation pressure may be applied to balloon 216 after compression (e.g., a compression head of the crimping apparatus may be held stationary when inflation pressure is applied) or, in some instances, inflation pressure may be applied to the balloon 216 before and/or during compression. The inflation pressure may be applied at about 10 psi to about 60 psi, or about 15 psi to about 40 psi, or about 20 psi to about 30 psi.

When inflation pressure is applied while the crimp head is stationary at a given diameter, the action of forming portions 228 of balloon 216 at least partially within some of the plurality of openings 226 may be primarily driven by the temperature and the inflation pressure used, as well as how open the openings 226 between the struts 224 are at the given diameter. If openings 226 are not open or are only partially opened, portions 228 may not protrude sufficiently into or through openings 226 to secure stent 218 to balloon 216. Openings 226 between struts 224 may vary based on manufacturing variables, so one process may not be ideal for all parts to be manufactured. To address manufacturing variables, for example, the processes disclosed herein may utilize compression steps that compress stent 218 to a number of different intermediate diameters and apply inflation pressure to balloon 216 while applying heat, as described herein. The processes disclosed herein may utilize an initial expansion step through balloon inflation before compressing the stent 218. For example, the crimping processes may compress stent 218 to a first intermediate diameter, to a second intermediate diameter, to a third intermediate diameter, to a fourth intermediate diameter, to a fifth intermediate diameter, etc., before the final crimped diameter of stent 218 is achieved. Inflation pressure may be applied before, during, and/or after any one or more of the compression steps.

Another example process may include the application of compressive force, heat, and inflation pressure, with a relatively slow compression rate, as described herein. In an example, the medical device is exposed to temperatures in a range of about 30 degrees Celsius (° C.) to about 80° C., such as, for example, above the glass transition temperature (T_{g}) of the balloon. The process may also include an expansion process through balloon inflation before the application of compressive force. This technique may allow stent 218 to be crimped from larger diameters, with a more open inflation channel/lumen 229 underneath stent 218, while maintaining sufficient stent retention forces (e.g., about 0.5 to about 1 pounds or about 0.75 pounds). During an expansion operation, balloon 216 may be formed up against the inner surface of stent 218 and portions 228 may protrude at least partially into or through openings 226 between stent struts 224 by simultaneous application of heat and balloon inflation pressure, while stent 218 is compressed at a relatively slow rate, as described herein. This technique enhances stent to balloon interaction by applying an inward radial compressive force on stent 218 into balloon 216, and an outward radial expansion force on balloon 216 at the same time, which may result in more engagement of portions 228 of balloon material with stent 218. The compression rate may be kept relatively slow, such as, for example, over about 15 seconds to about 120 seconds, about 30 seconds to about 60 seconds, or about 45 seconds, to allow sufficient balloon 216/stent 218 contact time for balloon 216 to be formed up against stent 218, and through stent struts 224 at each diameter (e.g., each intermediate diameter).

FIG. 4a is a graph that illustrates an example crimping process that may be similar to those disclosed herein. The graph includes a line showing changes to the diameter of the stent (e.g., stent 218) over time, generally labeled with reference number 300, and a line showing changes to the inflation pressure applied to the balloon (e.g., balloon 216) over time, generally bearing reference number 400. In this example and referencing stent 218 and balloon 216 of FIG. 2, stent 218 may have an initial diameter at an initial time period 301. The initial diameter may correspond to diameter D7M, D6E, D3E, or another diameter larger than D5I. In some instances, an initial expansion may occur (not shown) where stent 218 may be expanded from manufactured diameter D7M to a larger diameter (e.g., D6E, D3E, or another diameter larger than D7M). This may occur during time period 301 or prior to time period 301. Compression may begin at time period 302. Compression during time period 302 may be relatively slow (e.g., occurring over a time period of about 20 seconds to about 50 seconds or more, or about 25 seconds to about 40 seconds or more, or about 35 seconds to about 40 seconds or more). During the initial time period 301 and first compression time period 302 (e.g., at a time period 401), there may be no inflation pressure may applied to balloon 216. After completion of the first compression time period 302, inflation pressure may be increased (e.g., rapidly increased) at time period 402 and then held constant at time period 403. During a portion of time period 403, the diameter of stent 218 may be held constant (e.g., along time period 303). Inflation pressure may be decreased (e.g., rapidly decreased) at time period 404 and then held constant along time period 405. Along time period 405, the diameter of stent 218 may be reduced (e.g., along time period 304) and then held constant (e.g., along time period 305). The constant diameter at time period 305 may correspond to when stent 218 has diameter D5I. Inflation pressure applied during time period 405 may cause one or more portions portion 228 of balloon 216 to extend at least partially into openings 226.

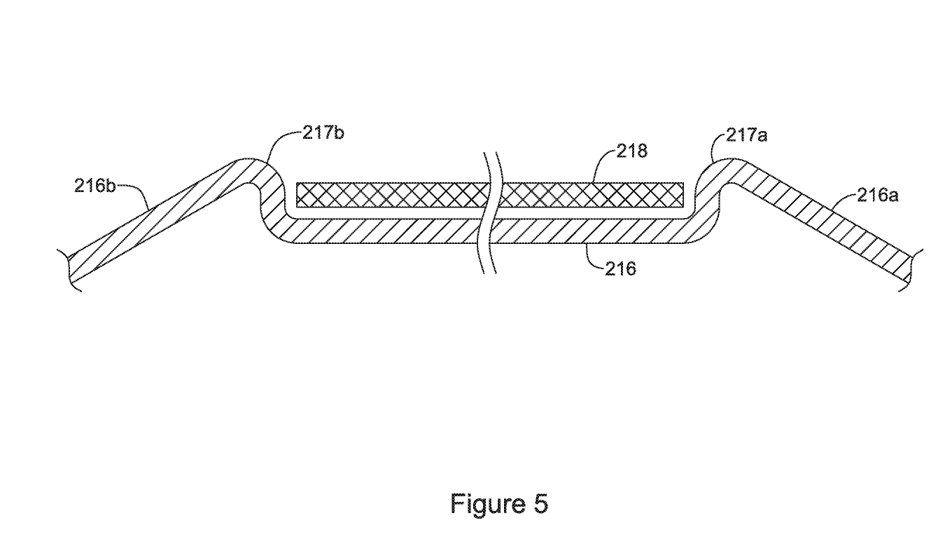

As shown in the example illustrated in FIG. 5, in at least some instances, portions of balloon 216 along a distal balloon cone 216a, a proximal balloon cone 216b, or both may also be formed up against stent 218. For example, a portion of balloon 216 at distal balloon cone 216a of balloon 216 may be formed up against the distal end of stent 218. This may define a ledge 217a that can further enhance securement of stent 218 to balloon 216. Such a ledge may be formed in addition to or instead of portions 228 of balloon 216 extending at least partially into or through openings 226 of stent 218. In some of these and in other instances, a portion of balloon 216 at proximal balloon cone 216b of balloon 26 may also form a ledge 217b.

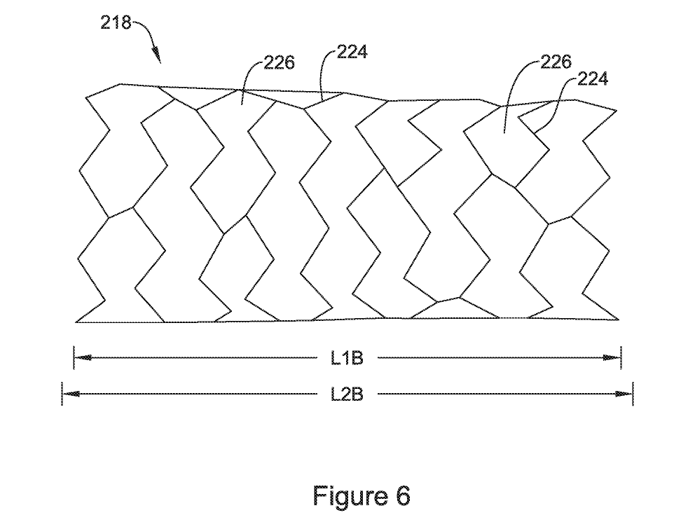

The processes disclosed herein may also help to control foreshortening. For example, as shown in FIG. 6, stent 218, having struts 224 and openings 226, may be fully expanded (e.g., at least foreshortened to a lesser degree that a single crimping process, such as referenced in relation to FIG. 2) such that the length L1B of stent 218 in an expanded configuration approximates the fully expanded length L2B. Because of the reduction in foreshortening, little or no strut overlap and/or deformation occurs.

In at least some instances, the implantable medical device disclosed herein may include a metal stent, a bioabsorbable metal stent, a drug coated metal stent, a drug coated bioabsorbable metal stent, a polymeric stent, a bioabsorbable polymeric stent, a drug coated polymeric stent, a drug coated bioabsorbable polymeric stent, or the like.

Some examples of suitable metallic materials that may be used for the metal stents contemplated herein may include iron, magnesium, manganese, platinum, chromium, nickel, cobalt, titanium alloys and/or combinations thereof, and the like, or other materials disclosed herein.

Some examples of suitable polymeric materials that may be used the polymeric stents contemplated herein may include poly-L-lactic acid (PLLA), poly-D-lactic acid (PDLA), poly-lactic acid (PLA), poly(lactic-co-glycolic acid) (PLGA), poly(ε-caprolactone) (PCL), desaminotyrosine polycarbonate and the like, or other materials disclosed herein.

Some examples of suitable drugs and/or therapeutic agents that may be used with the medical device contemplated herein may include paclitaxel and/or derivatives thereof, everolimus and/or derivatives thereof (e.g., the “limus” family of drugs), combinations thereof, and the like, or other suitable materials.

The materials that can be used for the various components of system 10 referenced in FIG. 1 (and/or other systems disclosed herein) may include those commonly associated with medical devices. For simplicity purposes, the following discussion makes reference to system 10. However, this is not intended to limit the devices and methods described herein, as the discussion may be applied to any of the systems/components disclosed herein, as appropriate.

System 10 and/or other components of system 10 may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “superelastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear that the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also can be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Some examples of nickel titanium alloys are disclosed in U.S. Pat. Nos. 5,238,004 and 6,508,803, which are incorporated herein by reference. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, portions or all of system 10 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of system 10 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of system 10 to achieve the same result.

In some embodiments, a degree of Magnetic Resonance Imaging (Mill) compatibility is imparted into system 10. For example, system 10, or portions thereof, may be made of a material that does not substantially distort the image and create substantial is artifacts (e.g., gaps in the image). Certain ferromagnetic materials, for example, may not be suitable because they may create artifacts in an MRI image. System 10, or portions thereof, may also be made from a material that the MM machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

### EXAMPLES

The following disclosure may be understood based on the following Examples, which are not intended to be limiting.

### Example 1

A drug-coated bioabsorbable polymeric stent was disposed about a balloon. The stent had a manufactured diameter of 0.13 inches and was slowly (e.g., a time period of about 30 seconds or longer) compressed to a diameter of 0.07 inches. After the compression step, inflation pressure (30 psi) was applied to the balloon for approximately 10 seconds. The stent was then slowly compressed (e.g., a time period of about 40 seconds or more) to a diameter of 0.04 inches. During this slow compression step, inflation pressure (10 psi) was applied. After the final compression step, an inflation pressure of 10 psi was applied for 60 seconds. Portions of the balloon were observed extending at least partially in and through the openings between the struts of the stent after the cycle was complete.

### Example 2

A metal stent was disposed about a balloon. The stent had a manufactured diameter of 0.08 inches and was slowly (e.g., a time period of about 15 seconds or more) expanded to a diameter of 0.11 inches. After the expansion step, inflation pressure (30 psi) was applied to the balloon for approximately 7 seconds. The stent was then slowly compressed (e.g., a time period of about 50 seconds or more) to a diameter of 0.05 inches. During this slow compression step, inflation pressure (10 psi) was applied. After the final compression step, an inflation pressure of 10 psi was applied for 20 seconds. Portions of the balloon were observed extending through the openings between the struts of the stent after the cycle was complete.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A method for preparing a medical device, the method comprising:
disposing an implantable medical device along an outer surface of a balloon;
wherein the implantable medical device includes a polymeric stent having a plurality of openings formed therein;
wherein the implantable medical device is designed to shift between a compressed configuration having a compressed diameter and an expanded configuration having an expanded diameter;
compressing the implantable medical device to an intermediate diameter between the compressed diameter and the expanded diameter; and
applying inflation pressure to the balloon so that at least a portion of the balloon extends at least partially into at least one of the plurality of openings in the implantable medical device.

2. The method of claim 1, further comprising expanding the implantable medical device to a diameter larger than a manufactured diameter of the implantable medical device before compressing the implantable medical device to the intermediate diameter.

3. The method of claim 1, further comprising applying heat to the implantable medical device.

4. The method of claim 3, wherein applying heat to the implantable medical device includes heating at a temperature of about 30° C. to about 80° C.

5. The method of claim 3, wherein applying heat to the implantable medical device includes heating at a temperature above the glass transition temperature (T_{g}) of the balloon.

6. The method of claim 3, wherein applying heat to the implantable medical device includes applying heat while compressing the implantable medical device.

7. The method of claim 6, wherein applying inflation pressure to the balloon occurs while compressing the implantable medical device.

8. The method of claim 6, wherein applying inflation pressure to the balloon occurs after compressing the implantable medical device.

9. The method of claim 1, further comprising compressing the implantable medical device to a second intermediate diameter that is smaller than the intermediate diameter.

10. The method of claim 1, wherein the implantable medical device is a bioabsorbable polymeric stent, a drug coated polymeric stent, or a drug coated bioabsorbable polymeric stent.

11. The method of claim 1, wherein applying inflation pressure to the balloon includes applying about 10 psi to about 60 psi of inflation pressure.

12. A medical device system, comprising:
an elongate shaft having a distal end region;
an expandable balloon coupled to the distal end region;
a stent secured to the balloon, wherein the stent has a plurality of openings formed therein;
wherein the stent is a polymeric stent, a bioabsorbable metal stent, or a drug coated metal stent; and
wherein at least a portion of the balloon extends at least partially into at least one of the plurality of openings in the stent.

13. The system of claim 12, wherein the stent is a bioabsorbable polymeric stent.

14. The system of claim 12, wherein the stent is a drug coated polymeric stent.

15. The system of claim 12, wherein the balloon has a distal balloon cone and a proximal balloon cone, and wherein a ledge is formed in the balloon adjacent to the distal balloon cone, the proximal balloon cone, or both, wherein the ledge is configured to enhance securement of the stent to the balloon.

16. A method for preparing a medical device, the method comprising:
disposing a stent along an outer surface of a balloon;
wherein the stent includes a plurality of openings;
wherein the stent is capable of shifting between a fully compressed configuration having a fully compressed diameter and an expanded configuration having an expanded diameter;
wherein the stent has a manufactured diameter that is between the fully compressed diameter and the expanded diameter;
expanding the stent from the manufactured diameter to a first intermediate diameter;
compressing the stent from the first intermediate diameter to a second intermediate diameter, the second intermediate diameter being smaller than the manufactured diameter and being between the fully compressed diameter and the expanded diameter;
applying inflation pressure to the balloon so that one or more sections of the balloon extend through at least some of the plurality of openings in the stent; and
applying heat to the stent while compressing the stent, while applying inflation pressure to the balloon, or while compressing the stent and applying inflation pressure to the balloon.

17. The method of claim 16, wherein applying heat to the stent includes applying heat while compressing the stent.

18. The method of claim 16, further comprising compressing the stent to a third intermediate diameter that is smaller than the second intermediate diameter.

19. The method of claim 16, wherein the stent is a metal stent, a bioabsorbable metal stent, a drug coated metal stent, or a drug coated bioabsorbable metal stent.

20. The method of claim 16, wherein the stent is a polymeric stent, a bioabsorbable polymeric stent, a drug coated polymeric stent, or a drug coated bioabsorbable polymeric stent.
